(19) United States
(12) Patent Application Publication
(10) Pub. No.: US 2023/0360220 A1
(43) Pub. Date: Nov. 9, 2023

(54) METHOD AND SYSTEM FOR IMAGING EYE BLOOD VESSELS

(71) Applicants: Ramot at Tel-Aviv University Ltd., Tel-Aviv (IL); Tel HaShomer Medical Research Infrastructure and Services Ltd., Ramat-Gan (IL)

(72) Inventors: Ygal ROTENSTREICH, Kfar-Bilu (IL); Ifat SHER ROSENTHAL, Shoham (IL); Haim SUCHOWSKI, Tel-Aviv (IL); Michael MREJEN, Tel-Aviv (IL); Shahar KATZ, Tel-Aviv (IL)

(73) Assignees: Ramot at Tel-Aviv University Ltd., Tel-Aviv (IL); Tel HaShomer Medical Research Infrastructure and Services Ltd., Ramat-Gan (IL)

(21) Appl. No.: 18/223,106

(22) Filed: Jul. 18, 2023

Related U.S. Application Data
(63) Continuation of application No. PCT/IL2022/050073, filed on Jan. 18, 2022.
(60) Provisional application No. 63/138,546, filed on Jan. 18, 2021.

(51) Int. Cl. G06T 7/00; A61B 5/026; A61B 3/11

(52) U.S. Cl. CPC G06T 7/0016; A61B 5/026; A61B 3/11; G06T 2207/10024; G06T 2207/20081; G06T 2207/30041; G06T 2207/30104

(57) ABSTRACT

A method of diagnosing a condition of a subject, comprises: receiving image data of an anterior of an eye of the subject, and analyzing the image data to detect at least one of: flow of individual blood cells in libmal or conjunctival blood vessels of the eye, morphology of limbal or conjunctival blood vessels. The method also comprises determining the condition of the subject based on the detection(s).

### RELATED APPLICATIONS

This application is a Continuation of PCT Patent Application No. PCT/IL2022/050073 having International filing date of Jan. 18, 2022, which claims the benefit of priority under 35 USC § 119(e) of U.S. Provisional Patent Application No. 63/138,546 filed on Jan. 18, 2021. The contents of the above applications are all incorporated by reference as if fully set forth herein in their entirety.

### FIELD AND BACKGROUND OF THE INVENTION

The present invention, in some embodiments thereof, relates to medical imaging and methods and, more particularly, but not exclusively, to a method and system for imaging eye blood vessels. Some embodiments of the present invention relates to diagnosis and optionally also prognosis of a disease, such as, but not limited to, COVID-19, leukemia, neutropenia due to high-dose chemotherapy, leukocytosis, polycythemia, anemia, low oxygen saturation. Some embodiments of the present invention relates to flow analysis of blood cells in the eye.

Known in the art are retinal imaging techniques that direct light into a patient's eye to illuminate a portion of the retina, and captures an image of the illuminated portion of the retina via light reflected off of the retina. Arichika et al. [Investigative Ophthalmology & Visual Science June 2013, Vol. 54, 4394-4402] discloses use of adaptive optics scanning laser ophthalmoscopy for acquiring videos from the parafoveal areas of an eye in order to identify erythrocyte aggregates. The erythrocyte aggregates are detected as dark tails that are darker regions than vessel shadows. The disclosed technique allows measuring time-dependent changes in lengths of the dark tails. Uji et al. [Invest Ophthalmol Vis Sci. 2012 Jan. 20; 53(1):171-8] discloses use of adaptive optics scanning laser ophthalmoscopy for acquiring videos from the parafoveal areas of the eyes. Gray-scale values inside and outside moving particles are measured and compared, and changes in the gray values of bright spots inside the capillaries are detected, before and during passage of the particles. The packing arrangements of the bright spots in the particles are analyzed, and the particle velocity is measured.

### SUMMARY OF THE INVENTION

According to some embodiments of the invention there is provided a method of diagnosing a condition of a subject. The method comprises: receiving a stream of image data of an anterior of an eye of the subject at a rate of at least 30 frames per second; applying a spatio-temporal analysis to the stream to detect flow of individual blood cells in limbal or conjunctival blood vessels of the eye; and, based on detected flow, determining the condition of the subject.

In some embodiments of the present invention the image data include at least one of: the cornea, the iris, the conjunctiva, the limbus, and episclera of the eye. In some embodiments of the present invention the image data include the eyelid of the eye.

According to some embodiments of the method comprises identifying hemodynamic and/or cardiovascular changes in the body of the subject based on the detected flow. According to some embodiments of the method comprises identifying local changes in the eye including hemodynamic and intraocular pressure. According to some embodiments of the method comprises determining a difference between the eyes.

According to some embodiments of the invention the spatio-temporal analysis comprises applying a machine learning procedure.

According to some embodiments of the invention the image data comprise at least one monochromatic image.

According to some embodiments of the invention the spatio-temporal analysis is selected to identify pupil light reflex events, wherein the determining the condition is based also on the identified pupil light reflex events.

According to some embodiments of the invention the spatio-temporal analysis is selected to detect to detect in the eye morphology of limbal or conjunctival blood vessels, wherein the determining the condition is based also on the detected morphology.

According to some embodiments of the invention the method comprises identifying flow of gaps.

According to some embodiments of the invention the method comprises measuring a size of the gaps.

According to some embodiments of the invention the method comprises measuring a flow speed of the gaps.

When the imaging is at a wavelength that identifies red blood cells, gaps can represent white blood cells preceded and followed by red blood cells.

According to some embodiments of the invention the flow is detected in at least two different vessels structures. According to some embodiments of the invention the at least two different vessels structures are selected from the group consisting of vessels of different diameters, and bifurcated vessels.

According to some embodiments of the invention the method comprises determining a density of the libmal or conjunctival blood vessels.

According to an aspect of some embodiments of the present invention there is provided a method of diagnosing a condition of a subject. The method comprises: receiving image data of an anterior of an eye; applying a spectral analysis to the image data to detect in the eye morphology of libmal or conjunctival blood vessels; based on the morphology, determining the condition of the subject.

According to some embodiments of the invention the image data comprises a set of monochromatic images, each being characterized by a different central wavelength.

According to some embodiments of the invention the image data is a stream of image data at a rate of at least 30 frames per second.

According to some embodiments of the invention the image data comprises at least one multispectral image.

According to some embodiments of the invention the multispectral images are characterized by a spectral range which comprises ultraviolet wavelengths (e.g., from about 10 nm to about 380 nm).

According to some embodiments of the invention the multispectral images are characterized by a spectral range which comprises visible wavelengths (e.g., from about 380 nm to about 780 nm).

According to some embodiments of the invention the multispectral images are characterized by a spectral range which comprises infrared (IR) wavelengths (e.g., from about 0.7 μm to about 1000 μm).

According to some embodiments of the invention the multispectral images are characterized by a spectral range which comprises near infrared (NIR) wavelengths (e.g., from about 780 nm to about 1030 nm).

According to some embodiments of the invention the multispectral images are characterized by a spectral range which comprises short-wavelength infrared (SWIR) wavelengths (e.g., from about 0.9 μm to about 2.2 μm).

According to some embodiments of the invention the multispectral images are characterized by a spectral range which comprises mid-wavelength infrared (MWIR) wavelengths (e.g., from about 2.2 μm to about 8 μm).

According to some embodiments of the invention the multispectral images are characterized by a spectral range which comprises long-wavelength infrared (LWIR) wavelengths (e.g., from about 8 μm to about 15 μm).

According to some embodiments of the invention the multispectral images are characterized by a spectral range which comprises far infrared (FIR) wavelengths (e.g., from about 15 μm to about 1000 μm).

According to some embodiments of the invention the multispectral images are characterized by a spectral range comprises visible and IR wavelengths.

According to some embodiments of the invention the multispectral images are characterized by a spectral range which comprises visible and NIR wavelengths.

According to some embodiments of the invention the multispectral images are characterized by a spectral range which comprises visible, NIR, and SWIR wavelengths.

According to some embodiments of the invention the multispectral images are characterized by a spectral range which comprises visible, NIR, SWIR and MWIR wavelengths.

According to some embodiments of the invention the multispectral images are characterized by a spectral range which comprises visible, NIR, SWIR, MWIR and LWIR wavelengths.

According to some embodiments of the invention the multispectral images are characterized by a spectral range which comprises visible, NIR, SWIR, MWIR, LWIR and FIR wavelengths.

According to some embodiments of the invention the multispectral images are characterized by a spectral range which comprises ultraviolet, visible, NIR, SWIR, MWIR, LWIR and FIR wavelengths.

According to some embodiments of the invention the method comprises capturing the image data.

According to some embodiments of the invention the method comprises executing an eye tracking procedure.

According to some embodiments of the invention the method comprises illuminating the eye by white light.

According to some embodiments of the invention the method comprises transmitting optical stimulus to the eye, before or during the capturing.

According to some embodiments of the invention the stimulus is monochromatic. According to some embodiments of the invention the stimulus is a blue stimulus. According to some embodiments of the invention the stimulus is a red stimulus. According to some embodiments of the invention the method comprises illuminating the eye by light at about 600-1000 nm.

According to some embodiments of the invention the method comprises measuring a density of the libmal or conjunctival blood vessels at two or more images of different wavelengths wherein the determining the condition is also based on the density. According to some embodiments of the invention the different wavelengths comprise a characteristic wavelength of melanin, a characteristic wavelength of oxygenated hemoglobin, a characteristic wavelength of deoxygenated hemoglobin, and/or a characteristic wavelength of methemoglobin.

According to an aspect of some embodiments of the present invention there is provided a method of diagnosing a condition of a subject. The method comprises: receiving input pertaining to a wavelength that is specific to the subject, and that induces pupil light reflex in a pupil of the subject; illuminating the pupil with light at the subject-specific wavelength; imaging an anterior of an eye of the subject at a rate of at least 30 frames per second to provide a stream of image data; applying a spatio-temporal analysis to the stream to detect pupil light reflex events; and based on detected pupil light reflex events, determining the condition of the subject.

According to some embodiments of the invention the condition is a disease. According to some embodiments of the invention the condition is a bacterial disease. According to some embodiments of the invention the condition is a viral disease. According to some embodiments of the invention the condition is a coronavirus disease.

According to some embodiments of the invention the condition is sepsis.

According to some embodiments of the invention the condition is a cardiac condition, or a cardio-vascular condition, e.g. heart failure.

According to some embodiments of the invention the condition is an ischemic condition.

According to some embodiments of the invention the condition is glaucoma.

According to some embodiments of the invention the condition is neuronal attenuation.

According to some embodiments of the invention the condition is a liver-related condition, e.g. jaundice.

According to some embodiments of the invention the condition is conjunctivitis.

According to some embodiments of the invention the method comprises generating an output describing the condition in terms of at least one parameter selected from the group consisting of a white blood cells count, red blood cells count, a platelets count, a hemoglobin level, an oxygenated hemoglobin level, a deoxygenated hemoglobin level, a methemoglobin level, a capillary perfusion, an ocular inflammation, blood vessel inflammation, venous return and blood flow.

According to some embodiments of the invention the method comprises providing prognosis pertaining to the condition.

According to an aspect of some embodiments of the present invention there is provided a system for diagnosing a condition of a subject. The system comprises an imaging system for capturing image data of an anterior of an eye of the subject; and an image control and processing system configured for applying the method as delineated above and optionally and preferably as further detailed below.

According to some embodiments of the invention the system comprises an eye tracking system.

According to some embodiments of the invention the system comprises a light source for transmitting an optical stimulus to the eye, before or during the capturing.

According to some embodiments of the invention the system comprises apparatus for fixation relative position between the eye and the imaging system.

According to some embodiments of the imaging system is hand held.

According to some embodiments of the invention the imaging system is a camera of a mobile device, and the image processing system is a CPU circuit of the mobile device.

According to some embodiments of the invention the imaging system is a camera of a mobile device, and the image processing system is remote from the mobile device.

According to some embodiments of the invention, the imaging system is portable, and include at least one functionality selected from the group consisting of autofocusing, interactive imaging algorithm, controllable shutter for increasing temporal resolution, adapted for allowing imaging in one or more of the aforementioned wavelength ranges.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of the method for determining a condition of a subject according to various exemplary embodiments of the present invention;

FIGS. 2A-C are schematic illustrations of a system suitable for executing the method described herein;

FIG. 3 is a block diagram of an exemplified eye imaging system according to some embodiments of the present invention;

FIG. 4 is a block diagram schematically illustrating a pipeline of an image processing procedure according to some embodiments of the present invention;

FIGS. 5A-F show results obtained in experiments performed according to some embodiments of the present invention on rabbit eyes;

FIGS. 6A-D show detection and tracking of blood cells in human conjunctival capillaries, as obtained in experiments performed according to some embodiments of the present invention;

FIGS. 7A-C show pupil contraction in a healthy volunteer before (FIG. 7A) and after (FIG. 7B) chromatic light stimulus, and attenuated pupil contraction FIG. 7C) in a subject having a brain tumor (red line, arrow) compared with age-similar controls (mean in a solid black line±SD in dashed lines) and its recovery following tumor removal (green line, block arrow), as obtained in experiments performed according to some embodiments of the present invention;

FIGS. 8A and 8B show correlation between red (FIG. 8A) and white (FIG. 8B) blood cell counts as obtained in experiments performed according to some embodiments of the present invention;

FIGS. 9A and 9B show additional correlation between red (FIG. 9A) and white (FIG. 9B) blood cell counts as obtained in experiments performed according to some embodiments of the present invention, where FIG. 9A shows Bland Altman analysis and FIG. 9B differentiates between leukemia patients (squares) and healthy subjects (circles); and

FIG. 10 is a block diagram of the system of the present embodiments in embodiments in which the system is used by an astronaut.

### DESCRIPTION OF SPECIFIC EMBODIMENTS OF THE INVENTION

The present invention, in some embodiments thereof, relates to medical imaging and methods and, more particularly, but not exclusively, to a method and system for imaging eye blood vessels. Some embodiments of the present invention relates to diagnosis and optionally also prognosis of a disease, such as, but not limited to, COVID-19, leukemia, neutropenia due to high-dose chemotherapy, leukocytosis, polycythemia, anemia, low oxygen saturation. Some embodiments of the present invention relates to flow analysis of blood cells in the eye.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

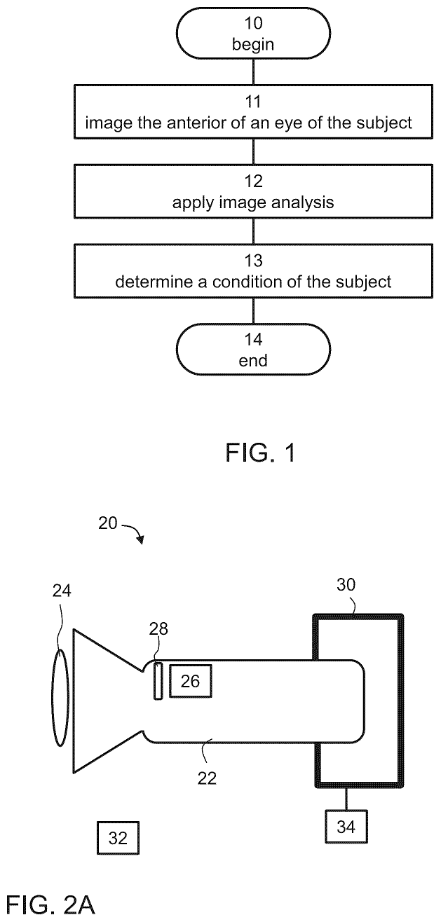

Referring now to the drawings, FIG. 1 is a flowchart diagram of the method according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be implemented by a data processing system, e.g., a dedicated circuitry or a general purpose processor, configured for executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pull these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer-readable medium, comprising computer-readable instructions for carrying out the method operations. It can also be embodied in electronic device having digital computer capabilities arranged to run the computer program on the tangible medium or execute the instruction on a computer-readable medium.

The method begins at 10 and optionally and preferably continues to 11 at which the anterior of an eye of the subject is imaged, to provide image data. The imaged region preferably comprises the conjunctiva and/or the limbus of the eye, and the imaging at 11 is preferably executed by ensuring that light reflected off the conjunctiva or the limbus is focused onto a sensor array of a camera. The focusing is optionally and preferably automatic by means of an autofocusing functionality of the camera. The autofocusing functionality is preferably embodied as dedicated circuitry and optics that are incorporated in the camera and that are specifically configured for autofocusing light reflected off the conjunctiva or the limbus, so as to ensure that a region in the image that includes the conjunctiva or the limbus appears sharper compared to other regions in the image.

The method can alternatively receive image data of the conjunctiva and/or the limbus of the eye from an external source, such as, but not limited to, a computer readable medium, or over a communication network. In this case, operation 11 can be skipped.

The image data (either obtained at 11 or received from the external source) can comprise one or more monochromatic images or it can comprise one or more multispectral images. In some embodiments of the present invention the image data comprise data acquired while or immediately after illuminating the eye by light having a wavelength that is specific to the subject and that induces pupil light reflex (PLR) in a pupil of the subject. Preferably, the image data comprise a stream of image data characterized by a rate of at least 30 frames per second. The image data can be captured by a camera mounted on a headset worn by the subject and being configured to place the camera in front of the eye of the subject. Alternatively, the image data can be captured by a portable hand-held camera. In some embodiments of the present invention the imaging 11 includes executing an eye tracking procedure. These embodiments are particularly useful when the image data are captured by a camera mounded on a headset.

In embodiments in which operation 11 is employed, the imaging is preferably executed under artificial illumination at one or more specific wavelengths within the visible range (e.g., from about 380 nm to about 780 nm), the near infrared (NIR) range (e.g., from about 780 nm to about 1030 nm), the short-wave infrared (SWIR) range (e.g., from about 0.9 μm to about 2.2 μm), the long-wavelength infrared (LWIR) range (e.g., from about 8 μm to about 15 μm), and/or the far infrared (FIR) range (e.g., from about 15 μm to about 1000 μm). Also contemplated, are embodiments in which the imaging is executed under artificial illumination at one or more specific wavelengths within the ultraviolet range (e.g., from about 10 nm to about 380 nm).

The imaging is preferably by one or more digital cameras that are sensitive to these wavelengths. Representative examples include, without limitation, a CMOS camera (e.g., a NIR-filtered visible light CMOS camera), a NIR enabled CMOS camera, and an uncooled InGaAsP camera. In some embodiments of the present invention the imaging is by a hyperspectral CMOS SWIR camera.

The specific wavelengths are preferably selected in accordance with the typical optical properties of blood components in blood vessels within the eye.

For example, the absorption spectra of red blood cells (RBCs) is dominated by the optical properties of hemoglobin, and so the specific wavelength(s) is/are optionally and preferably selected to generate sufficient contrast at image regions that correspond to eye regions dominated by hemoglobin, so as to identify RBC. Further, the specific wavelengths can include a distinct wavelength within the absorption spectrum of oxygenated hemoglobin, which is typically at about 600-700 nm, and another distinct wavelength within the absorption spectrum of deoxygenated hemoglobin, which is typically at about 900-1000 nm, thus providing image data that distinguish between regions or frames that contain oxygenated hemoglobin, and regions or frames that contain deoxygenated hemoglobin. Such image data can be used to determine saturation of peripheral oxygen. Also contemplated, are embodiments in which the specific wavelengths include a distinct wavelength within the absorption spectrum of methemoglobin (MetHb), which is typically at about 600-650 nm, with a peak at about 630 nm. The advantage of these embodiments is that they allow identifying a toxic condition for the subject under analysis.

Unlike RBCs, white blood cells (WBCs) have a peak absorbance in infrared (IR) and ultraviolet (UV) ranges, and so the specific wavelength(s) is/are optionally and preferably selected within the IR and/or UV ranges to generate sufficient contrast at image regions that correspond to eye regions dominated by WBCs. Another blood component for which contrast can be generated by a judicious selection of the specific wavelength(s) include platelets. The peak absorbance of platelets is about 450 nm and about 1000 nm, and so and so the specific wavelength(s) is/are optionally and preferably selected at about 450 nm and/or about 100 nm to generate sufficient contrast at image regions that correspond to eye regions dominated by platelets.

It is appreciated that the desired illumination wavelength(s) can be ensured either by selecting a wavelength specific light source, or by selecting a broadband light source (for example, white light) in combination with a set of bandpass filters. In some embodiments of the present invention, the illumination is continuous and in some embodiments of the present invention the illumination is in flashes. Flashes are preferred when the imaging generate a stream of image data since it allows reducing the effective duration per frame. For example, use of flashes at a duration of about 5 ms per flash, in combination of a frame rate of about 30 frames per seconds, can reduce the exposure time from about 30 ms per frame to about 1 ms per frame.

In some embodiments of the present invention the imaging 11 comprises transmitting an optical stimulus to the eye, before and/or during the image capture. The stimulus can be monochromatic, for example, a blue stimulus or a red stimulus so as to induce neuroretinal responses. The advantage of these embodiments is that they allow detecting attenuated neuronal function. Preferably, the optical stimulus is applied over a duration of less than 1 seconds (e.g., from about 300 ms to about 700 ms), or applied repeatedly in pulses having a pulse width of less than 1 seconds (e.g., from about 300 ms to about 700 ms). In some embodiments of the present invention the method receives input pertaining to a wavelength that is specific to the subject, and that induces PLR in the pupil of the subject, in which case the stimulus is applied at this subject-specific wavelength.

The method proceeds to 12 at which image analysis is applied to the image data. The type of image analysis depends on the type of image data obtained at 11 or received from the external source. Specifically, when the image data are multispectral, the image analysis comprises spectral analysis, and when the image data include a stream of image data, the image analysis comprises a spatio-temporal analysis. It is appreciated that combinations of these types of analyses is also contemplated as the case may be. For example, when the image data include a stream of multispectral image data, the image analysis can comprise spectral spatio-temporal analysis. On the other hand, when the image data include a stream of monochromatic image data, there is no need for performing the analysis over the spectral domain, and when the image data is not in the form of a stream (e.g., one or more distinct still images) there is no need for performing the analysis over the temporal domain.

The image analysis can include one or more image processing procedures. For example, the data can be processed to detect a morphology of libmal or conjunctival blood vessels in the eye, and more preferably to identify changes in the scleral and conjunctiva blood vessel morphology following conjunctivitis induction. When the image data include a stream of image data, the image processing procedure can be spatio-temporal so as to identify blood flow, and more preferably changes in blood flow following conjunctivitis induction. Also contemplated, are embodiments in which the spatio-temporal image processing procedure identifies PLR events. These embodiments are particularly useful when the illumination is at one or more wavelengths that induce PLR, which wavelengths can be either typical to a group of subjects, or be subject-specific.

The image processing procedure can also be used for tracking individual RBCs traveling through capillaries of the vasculature having a diameter of less than 30 μm, e.g., from about 10 μm to about 20 μm. In such capillaries, each WBC generally occupies the entire width of the capillary, and the image processing procedure can additionally or alternatively be used for tracking flow of gaps within capillaries, wherein each such gap can correspond to a WBC region between two individual RBCs. The image processing procedure can measure the flow speed and/or size of such gaps. The measured size can optionally and preferably be used for estimating the number of WBCs within each gap, and the measured speed can optionally and preferably be used for determining the mobility of the WBCs in the capillaries.

The image processing procedure can also be used for detecting flow in two or more different vessels structures. For example, a flow can be detected in blood vessels of different diameters. A flow can also be detected in bifurcated vessels.

In some embodiments of the present invention the image processing procedure determines the density of the limbal or conjunctival blood vessels. Such density can be used to estimate the condition of the eye, for example, following conjunctival induction. Specifically, when the density is higher close to the limbus but lower towards the posterior parts of the eye, the method can determine that the eye's condition is likely to be normal, and when the density is low close to the limbus but high across other regions the method can determine that it is likely that the eye experienced conjunctival induction.

The image processing procedure can include any of the image processing procedures known in the art, including, without limitation, image alignment, image stitching, and one or more low-level operations, e.g., undistort, gamma-correction, and the like. Image alignment is the process of matching one image to another on the spatial domain. In some embodiments of the present invention image alignment is executed to compensate for motions of the eye between successive frames. Image stitching is the process of combining overlapping images to get a larger field of view, and is preferably executed when the field-of-views of two or more of the images differ.

In some embodiments of the present invention image processing procedure includes a machine learning procedure.

As used herein the term “machine learning” refers to a procedure embodied as a computer program configured to induce patterns, regularities, or rules from previously collected data to develop an appropriate response to future data, or describe the data in some meaningful way.

In machine learning, information can be acquired via supervised learning or unsupervised learning. In some embodiments of the invention the machine learning procedure comprises, or is, a supervised learning procedure. In supervised learning, global or local goal functions are used to optimize the structure of the learning system. In other words, in supervised learning there is a desired response, which is used by the system to guide the learning.

In some embodiments of the invention the machine learning procedure comprises, or is, an unsupervised learning procedure. In unsupervised learning there are typically no goal functions. In particular, the learning system is not provided with a set of rules. One form of unsupervised learning according to some embodiments of the present invention is unsupervised clustering (e.g. backgrounds and targets spectral signatures and special characteristics) in which the data objects are not class labeled, a priori.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, artificial neural networks, instance-based algorithms, linear modeling algorithms, k-nearest neighbors analysis, ensemble learning algorithms, probabilistic models, graphical models, logistic regression methods (including multinomial logistic regression methods), gradient ascent methods, singular value decomposition methods and principle component analysis. Among neural network models, the self-organizing map and adaptive resonance theory are commonly used unsupervised learning algorithms. The adaptive resonance theory model allows the number of clusters to vary with problem size and lets the user control the degree of similarity between members of the same clusters by means of a user-defined constant called the vigilance parameter.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification or a value. The leaf nodes can also contain additional information about the represented classification such as a confidence score that measures a confidence in the represented classification (i.e., the likelihood of the classification being accurate). For example, the confidence score can be a continuous value ranging from 0 to 1, which a score of 0 indicating a very low confidence (e.g., the indication value of the represented classification is very low) and a score of 1 indicating a very high confidence (e.g., the represented classification is almost certainly accurate).

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regressions also include a multinomial variant. The multinomial logistic regression model, is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.).

Artificial neural networks are a class of algorithms based on a concept of inter-connected computer program objects referred to as neurons. In a typical artificial neural network, neurons contain data values, each of which affects the value of a connected neuron according to a pre-defined weight (also referred to as the “connection strength”), and whether the sum of connections to each particular neuron meets a pre-defined threshold. By determining proper connection strengths and threshold values (a process also referred to as training), an artificial neural network can achieve efficient recognition of image features. Oftentimes, these neurons are grouped into layers in order to make connections between groups more obvious and to each computation of values. Each layer of the network may have differing numbers of neurons, and these may or may not be related to particular qualities of the input data. An artificial neural network having a layered architecture belong to a class of machine learning procedure called “deep learning,” and is referred to as deep neural network (DNN).

In one implementation, called a fully-connected DNN, each of the neurons in a particular layer is connected to and provides input value to those in the next layer. These input values are then summed and this sum is compared to a bias, or threshold. If the value exceeds the threshold for a particular neuron, that neuron then holds a value which can be used as input to neurons in the next layer of neurons. This computation continues through the various layers of the neural network, until it reaches a final layer. At this point, the output of the DNN can be read from the values in the final layer.

Unlike fully-connected DNNs, convolutional neural networks (CNNs) operate by associating an array of values with each neuron, rather than a single value. The transformation of a neuron value for the subsequent layer is generalized from multiplication to convolution. When the neural network is a CNN, the training process adjusts convolutional kernels and bias matrices of the CNN so as to produce an output that resembles as much as possible known image features.

The final result of the training of an artificial neural network having a layered architecture (e.g., DNN, CNN) is a network having an input layer, at least one, more preferably a plurality of, hidden layers, and an output layer, with a learn value assigned to each component (neuron, layer, kernel, etc.) of the network. The trained network receives an image at its input layer and provides information pertaining to images feature present in the image at its output layer.

The training of an artificial neural network includes feeding the network with training data, for example data obtained from a cohort of subjects. The training data include images which are annotated by previously identified image features, such as regions exhibiting pathology and regions identified as healthy. Based on the images and the annotation information the network assigns values to each component of the network, thereby providing a trained network. Following the training, a validation process may optionally and preferably be applied to the artificial neural network, by feeding validation data to the network. The validation data is typically of similar type as the training data, except that only the images are fed to the trained network, without feeding the annotation information. The annotation information is used for validation by comparing the output of the trained network to the previously identified image features.

In embodiments in which a trained machine learning procedure is employed, the procedure is fed with the image data, and the trained machine learning procedure generates an output indicative of the condition of the eye.

The output of the machine learning procedure can be a numerical output, for example, a numerical output describing a WBCs count, a RBCs count, a platelets count, a hemoglobin level, an oxygenated hemoglobin level, a deoxygenated hemoglobin level, a methemoglobin level, a capillary perfusion, an ocular inflammation level, a blood vessel inflammation level, and/or a blood flow.

The output of the machine learning procedure can additionally or alternatively include a classification output. For example, the output can indicate whether the condition of the subject is considered as healthy or unhealthy or suffering from a particular disease, or be in the form of a score (for example, a [0,1] score) indicative of the membership level of the subject under investigation to a particular classification group (e.g., a classification group of healthy subjects, a classification group of unhealthy subjects, a classification group of subjects suffering from a particular disease, etc.) The classification output can be associated with a specific parameter (e.g., normal or abnormal WBCs count, normal or abnormal RBCs count, normal or abnormal platelets count, normal or abnormal hemoglobin level, normal or abnormal oxygenated hemoglobin level, normal or abnormal deoxygenated hemoglobin level, normal or abnormal methemoglobin level, normal or abnormal capillary perfusion, normal or abnormal ocular inflammation level, normal or abnormal blood vessel inflammation level, and/or normal or abnormal blood flow), or it can be a global classification output that weighs one or more such parameters.

The use of machine learning can be instead of the other image processing procedures described above, or more preferably in addition to one or more other image processing procedures. For example, procedures such as image alignment, image stitching, and other low-level operations, can be applied for enhancing selected features in the images, and the machine learning can be applied to the enhanced images, for example, for the purpose of feature extraction and classification.

The method continues to 13 at which the condition of the subject is determined based on the analysis. The determined condition can be displayed on a display device, and/or transmitted to a local or remote computer readable medium, or to a computer at a remote location. Typically, the method identifies hemodynamic changes in the body of the subject. In some embodiments of the present invention the method identifies changes in WBC, for example, based on the identified gaps in libmal or conjunctival capillaries, and in some embodiments of the present invention the method identifies attenuated neuronal function based on the identified PLR, e.g., following the application of optical stimulus. Preferably, the method generates an output describing the condition in terms of WBCs count, RBCs count, platelets count, hemoglobin level, oxygenated hemoglobin level, deoxygenated hemoglobin level, methemoglobin level, capillary perfusion, ocular inflammation, blood vessel inflammation, blood flow, and the like.

The condition determined at 13 can in some embodiments of the present invention is typically a condition that affects the hemodynamics of the subject. The likelihood that the subject has such a condition can be determined based on the identified hemodynamic changes. The changes can be relative to a baseline that is specific to the subject or to a baseline that is characteristic to a group of subjects. Thus, the method can access a computer readable medium storing data pertaining to the hemodynamics of the specific subject, or data pertaining to the characteristic hemodynamics of a group of healthy subjects, and use the stored data as the baseline for determining the changes.

Representative examples for conditions that can be determined by the method include, without limitation, a disease, for example, leukemia, neutropenia, anemia, polycythemia, a bacterial disease, or a viral disease, e.g., a coronavirus disease, such as, but not limited to, SARS-CoV-2, sepsis, a heart failure, an ischemic condition, glaucoma, neuronal attenuation, jaundice, conjunctivitis.

In some embodiments of the present invention the method provides prognosis pertaining to the condition. Such a prognosis can be based on the extent of hemodynamic changes and on the group of subjects to which the subject belongs.

The method ends at 14.

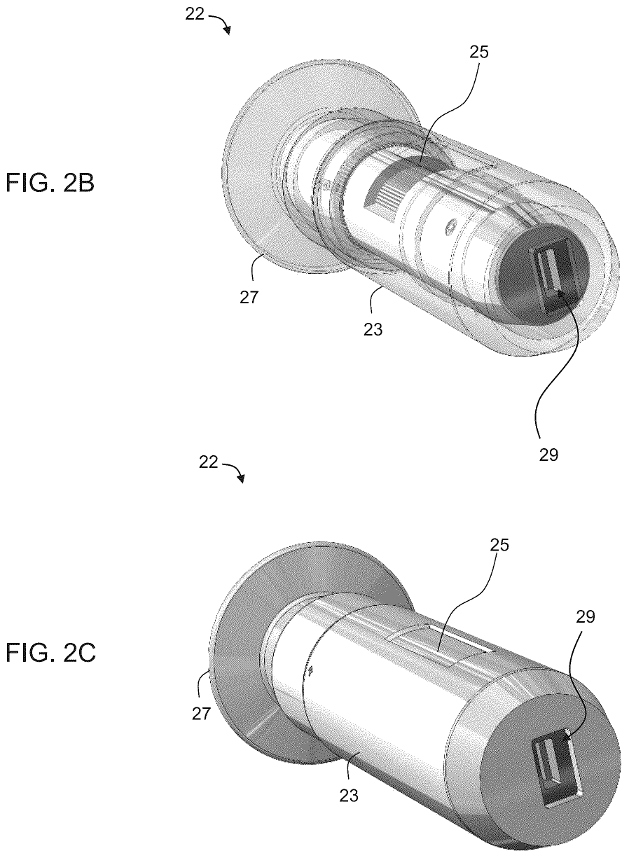

A schematic illustration of a system 20 suitable for executing the method of the present embodiments is illustrated in FIGS. 2A-C. System 20 can comprise an imaging system 22 for capturing image data of the anterior of an eye 24 of the subject (not shown). Imaging system 22 can include any of the aforementioned cameras. According to some embodiments, the imaging system 22 includes at least one functionality selected from the group consisting of autofocusing, and controllable shutter for increasing temporal resolution. Imaging system 22 is preferably selected for allowing imaging in one or more of the aforementioned wavelength ranges.

In some embodiments of the present invention imaging system 22 comprises one or more light sources 26 for illuminating and/or stimulating the eye as further detailed hereinabove, and may optionally and preferably also include a set of filters 28 for filtering the generated light as further detailed hereinabove. In some embodiments of the present invention system 20 comprises apparatus 30 for fixation relative position between eye 24 and imaging system 22. Apparatus 30 can be mounted on a headset (not shown) worn by the subject. In some embodiments of the present invention system 20 comprises an eye tracking system 32 configured for tracking a gaze of the subject.

Imaging system 22 can, in some embodiments of the present invention be a hand held system. A representative example of a hand held configuration for system 22 is schematically illustrated in FIGS. 2B and 2C. Shown is imaging system 22 having an encapsulation 23 provided with a service window 25 and an eye piece 27 configured to interface with eye 24 (not shown) to prevent ambient light from entering the encapsulation 23 while system 22 captures an image of the conjunctiva or the limbus of eye. Encapsulation 23 can encapsulate the sensor array and the optics of system 22, as well as the light source 26, the set of filters 28, and the autofocusing functionality (not shown) that ensures focused of light reflected off the conjunctiva or the limbus. System 22 typically includes also a power and data port 29, such as, but not limited to, a universal serial bus (USB) or the like.

System 20 can further comprise an image control and processing system 34 configured for controlling imaging system 22 and for applying various operations of the method described herein. While image control and processing system 34 is illustrated as a single component, it is appreciated that such a system can be provided as more than one components. For example, image control and processing system 34 can include an image control system that is separated from the image processing system. The image control system and the image processing system can in some embodiments of the present invention be remote from each other. In some embodiments, the image control system receives gaze information from tracking system 32 and controls apparatus 30 to reposition imaging system 22 responsively to the received gaze information.

In some embodiments of the present invention imaging system 22 is a camera of a mobile device, such as, but not limited to, a smartphone or a tablet, of a laptop computer, in which case at least part of the image processing is executed by the CPU circuit of the mobile device. Alternatively, or additionally, at least part of the image processing is executed remote from imaging system 22.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

White-blood-cell (WBC) count is employed for numerous clinical procedures as one of the indicators for immune status, mainly in patients undergoing chemotherapy or other immunosuppressant treatments, patients with leukemia, sepsis, infectious diseases, and autoimmune disorders. Currently, WBC counts are determined by clinical laboratory analysis of blood samples. Blood sample collection is invasive and necessitates visits to medical centers. Sterile conditions and qualified personnel are required for the blood sample analysis, limiting the accessibility and frequency of the measurement.

The Inventors realized that these limitations can interfere patients' care, for example, limiting timely life-saving interventions in afebrile patients with prolonged severe neutropenia. The Inventors also realized that it is advantageous to minimize visits to clinics or hospitals by patients undergoing chemotherapy so as to prevent infections.

The outbreak of Covid 19 pandemic placed patients with cancer at high risk for lethal complications of the disease, and in certain countries quarantines prevented patients to reach to medical centers. The Inventors have therefore devised a non-invasive technique for monitoring WBC count. The WBC count technique according to some embodiments of the present invention can be done quickly and in some embodiments by telemedicine, for example, from home.

In narrow capillaries of the vasculature, the capillary diameter approaches WBC diameter (10-20 μm). Hence, the WBC fill the capillary lumen. Since the velocity of WBCs is slower than that of red blood cells (RBCs), a “depletion” of RBCs occurs downstream of the WBC in the microcirculation. The Inventors found that illumination of blood vessel with light can allow detecting RBCs that look dark as they absorb the light, whereas WBCs stay transparent. The passage of a WBC in narrow capillaries of the vasculature thus appears as an optical absorption gap in the continuous “dark” RBC stream that moves through the capillary. This was shown in rabbit ears using white light as well as in rat-cremaster, bat-wings and human nailfold capillaries, using blue light transillumination to maximize the contrast between high absorbing RBCs (that carry large amounts of hemoglobin that has maximal light absorption at 420 nm) and low-absorption regions lacking RBCs.

Since the skin typically masks narrow capillaries of the vasculature imaging of human nailfold capillaries is difficult to perform in subjects having these tiny vessels, hence this method can be used only in people with skin color characterized by a Fitzpatrick scale of 4 or above. Absorption gaps can be perceived by subjects as blue spots when their eyes are illuminated with blue light and they are asked how many bright spots they see: the WBCs pass the blue light which is perceived by the subjects as blue dots. The amounts of perceived spots varies between baseline (normal), leukopenic (abnormally low), and leukocytotic (abnormally high WBC counts) subjects. The absorption gaps were observed in human retina vessels by adaptive optics scanning laser ophthalmoscopy.

The COVID-19 pandemic presents an unprecedented global health crisis that is leading to the greatest economic, financial and social shock of the 21′ Century. Beyond the obvious necessity of a vaccine, curbing this pandemic requires urgently a quick, cheap and accessible tool for COVID-19 diagnosis. Currently COVID-19 diagnosis involves the collection of nasopharyngeal swabs followed by RT-PCR analysis. The inventors appreciate that this procedure is time consuming, costly, and requires maintenance of sterile conditions, expensive equipment and highly qualified personnel. The test cannot be performed frequently, and its results are obtained only after several hours or even days. The inventors have therefore searched for real-time sensitive diagnosis tools for COVID-19.

Furthermore, one of the disease characteristics is the sudden deterioration of mild and moderate patients which may lead to mortality. The inventors have therefore developed efficient and sensitive indicators for disease prognosis to shorten the path to therapeutic response and reduce the mortality rate.

Blood lymphocyte percentage represents a possible reliable indicator to the criticality of COVID-19 patients. However, the inventors appreciate that it requires blood tests that have similar shortcomings as for the case of collecting nasopharyngeal swabs. Recent studies reported that 31.6% of COVID-19 patients have ocular manifestations of conjunctivitis including chemosis, conjunctival hyperemia, epiphora, or increased secretions. Patients with ocular symptoms were more likely to have higher white blood cell (WBC) and neutrophil counts than patients without ocular symptoms. 91% of these patients were positive for SARS-CoV-2 on RT-PCR from nasopharyngeal swabs. The inventors have therefore developed a noninvasive and real-time COVID-19 diagnosis that is based on imaging of the eye.

COVID-19 patients present with neurologic symptoms, such as loss of smell and taste, dizziness, headaches and nausea, and loss of smell and taste may present a strong predictor for COVID-19. The inventors appreciate that the self-report nature of this measure limits its use in clinical evaluation.

Pathology studies indicate that the virus invades the central nerve system (CNS), and its functional receptor (ACE2) is highly expressed in the brain and retina, including the retinal photoreceptors and ganglion cells, whose axons form the optic nerve that connects the retina and the brain. The retina is part of the CNS and is often considered a “window to the brain”, as neuronal, vascular and immunological changes in the brain can be easily identified in the retina. The inventors therefore contemplate that attenuated pupil light reflex (PLR) can serve as a sensitive early diagnostic tool for COVID-19.

This Example describes a real-time on-the-spot sensitive platform for COVID-19 diagnosis and prognosis based on sensitive high-resolution multispectral imaging of the anterior part of the eye in the Visible-Near-infrared. The system described herein is configured for detecting at least one of: (i) subtle changes in the libmal or conjunctival blood vessel morphology at various wavelengths, typically associated with conjunctivitis; (ii) changes in WBC counts based on optical-absorption gaps in the libmal or conjunctival capillary lumen; and (iii) attenuated neuronal function by high resolution tracking of the PLR for very short (e.g., about 500 ms) red and blue light stimuli to assess changes in various neuroretinal pathways.

The system described herein is based on the simultaneous detection of systemic changes in neural, immune and vascular systems via rapid imaging of the anterior segment of the eye, and can provide real-time (e.g., within seconds to minutes), sensitive and specific noninvasive test for COVID-19 diagnosis and prognosis. The system can be used frequently and quickly for continuous monitoring of patients deterioration or recovery. The system can be implemented as a small portable imaging device (e.g., a smartphone phone) for use in community clinics, entrance to shopping-centers and at home via telemedicine.

The system described herein can assist in decision making regarding COVID-19 patients care in medical centers, confinement and isolation. The system can optionally and preferably also be used in telemedicine-based real-time, non-invasive and continuous assessment of WBC counts, hemoglobin levels, capillary perfusion, ocular inflammation, and neuronal attenuation for patients with other diseases (e.g. neurodegeneration, sepsis, patients undergoing chemotherapy, critically ill patients etc.).

### Materials and Methods

Animals—20 rabbits (10 males & 10 females) were purchased from Envigo (Rehovot, Israel). All animal procedures and experiments were conducted with approval and under the supervision of the Institutional Animal Care Committee at the Sheba Medical Center, Tel-Hashomer, Israel, and conformed to recommendations of the Association for Research in Vision and Ophthalmology Statement for the Use of Animals in Ophthalmic and Vision Research. Rabbits underwent multimodal multispectral imaging before and following induction of conjunctivitis by injection of Complete Freund's Adjuvant to the superior eyelid. This model was chosen as it closely mimics the conjunctivitis symptoms seen in patients.

Imaging System—A high speed (more than 150 frames per second) multispectral imaging system is used for retrieving high resolution eye scans at VIS-NIR spectral range allowing both spectral and spatial information retrieval. The high speed acquisition ensures faithful capturing of blood flow in vessels. In conjunction with adequate eye tracking algorithms, it also allows detection of abnormal pupil light reflex (PLR) associated with affected neurological activity.

Hardware components Multimodal imaging: still images and video imaging.

Wide range of light sources for ocular imaging: combining visible 380 [nm] to 780 [nm], near infrared (NIR) from 780 [nm] to 1030 [nm] and short-wave infrared (SWIR) 0.9 [um]−2.2 μm] wavelength range, and a hyperspectral CMOS/SWIR camera.

Several bandpass filters (including a filter at about 777 nm for oxygen level, and a bandpass filter of about 620-720 nm for Melanin level) for accurate spectral sectioning. The illumination intensities and duration of the sources do not exceed the safety standard for clinical use.

The digital unit houses a sNear IR filtered, CMOS (visible camera), a fast (hundreds of frames per second) Near IR enabled (up to 1.03 um (wavelength)) and an uncooled InGaAsP (sensitive to 2.2 um).

Filters for improved images at various wavelengths. For example: blue light stimulus with yellow filter.

Autofocus for automatic high resolution images, compensating for eye movements (saccades etc.).

Eye tracking system.

Evaluation of different parts of the eye will be feasible by different fixation points.

High speed camera.

A computer (and software) for controlling the camera.

Background light including infrared light source.

Automatic control for light illumination duration and intensity.

Closed compartment for dark adaptation and controlling background light intensity.

Device for fixation of eye position (helmet/chin and forehead rest).

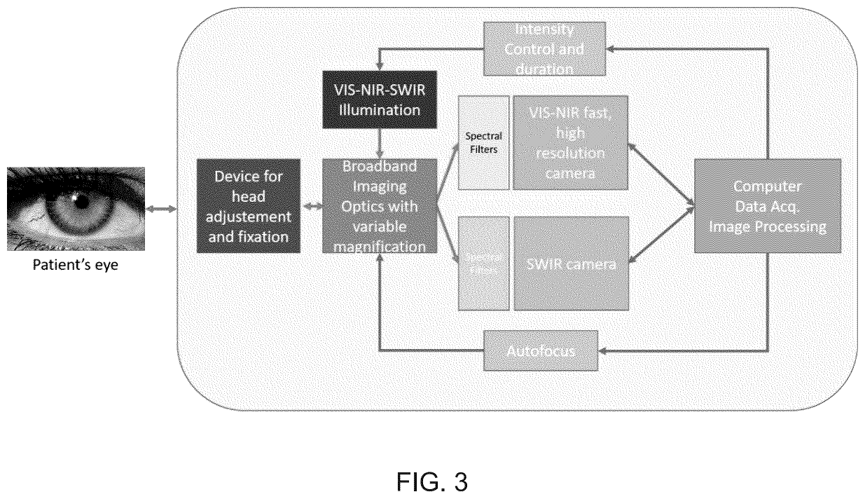

A block diagram of the eye imaging system of this Example is shown in FIG. 3. Each sensor captures images in a specific spectral range with timestamp for tracking and comparison. The acquired images, once acquired, are subjected to image processing described below.

Data Analysis

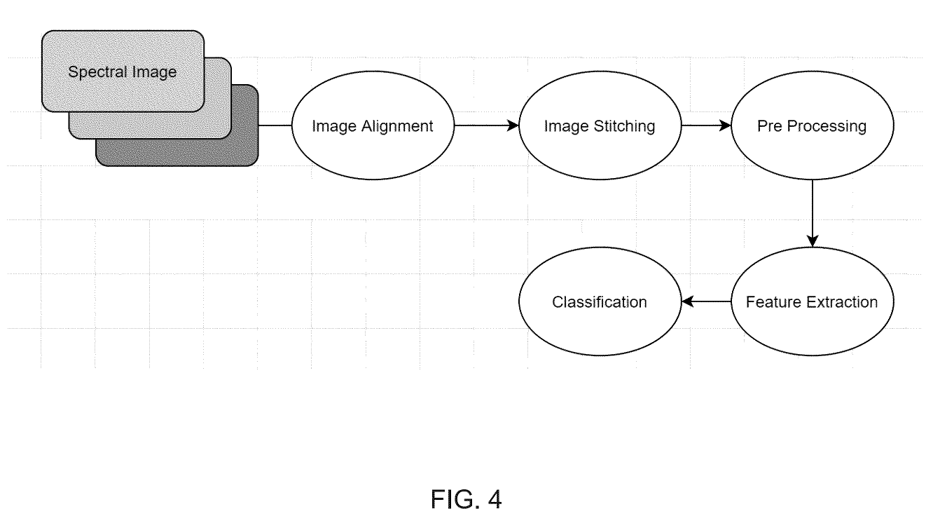

The data was analyzed in two phases. First, advanced image processing was applied for retrieval of subtle changes in the scleral and conjunctiva blood vessel morphology and blood flow following conjunctivitis induction. The use of such analysis in several spectral lines allows the differentiation of various biological markers (such as oxygen, red- and white-blood cell densities, etc.). In a second phase, a machine learning procedure was applied for detection and classification. The pipeline of the image processing included five main blocks shown in FIG. 4.

Image Alignment is the process of matching one image to another on the spatial domain. The purpose of the image alignment was to compensate moving objects in the scene, moving scenes or images from different points of view, such as images from two cameras.

Image Stitching, is the process of combining overlapping images to get a larger field of view.

The pre-processing can optionally and preferably include more than one low-level operation such as, but not limited to, undistort, gamma-correction, and the like. Preferably, pre-processing is applied to enhance image some features and/or suppress distortions.

Feature Extraction can be applied to extract from the images one or more features, such as, but not limited to, blood cells, blood vessels shape, iris spectrum, blood cells spectrum, movement speed of blood cell. In some embodiments of the present invention feature extraction can be executed by the machine learning procedure.

Classification can be applied to classify the subject according to one or more of sick, healthy, blood oxygen level, hemoglobin level, white blood cell count. The classification can be performed using any machine learning procedure, such as, but not limited to, Decision Tree (DT), Logistic Regression (LS), Support Vector machine (SVM), Deep Neural Network (DNN), and the like.

The system of the present embodiments can use several filters for further spectral sectioning to observe changes in spatial-morphological and temporal-morphological changes. Examples include filters to observe oximetry in the blood vessels of the eye (such as 777 nm or other wavelengths) or filters to observe melanin.

The system optionally and preferably has an automatic slit control, automatic light power emission, and automatic focusing for better quantifications of the temporal and morphological changes for different wavelengths.

The system optionally and preferably has automatic selection of light power emission and/or focusing, so as to facilitate extraction of morphological and/or temporal features.

The system optionally and preferably has a headset or helmet with goggles that includes magnifications and automatic control (focusing, spectral filters) to allow capturing the images for the analysis.

### Results

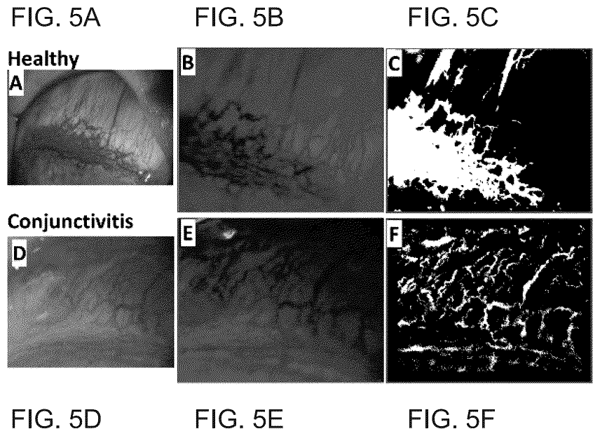

Images of rabbit eyes were analyzed for changes in the capillary network before and following conjunctivitis induction. The results are shown in FIGS. 5A-F, for the healthy (FIGS. 5A-C) and conjunctivitis-induced (FIGS. 5D-F) rabbit eyes. FIGS. 5C and 5F show processed images where white color indicates the blood vessels network. As observed in the processed image (FIG. 5F), the white region is significantly less pronounced in the conjunctivitis eye, leading to a distribution that is dramatically more uniform than in the healthy eye (FIG. 5C).

FIGS. 5A-F thus show substantial differences in density and distribution of the conjunctival blood vessel network following conjunctival induction. In the heathy rabbit, the network is dense close to the limbus but sparse towards the posterior parts. In contrast, following conjunctivitis induction the density and distribution of the blood vessel decreased significantly close to the limbus, but increased across the entire eye, which is the main reason it is observed as a “red-eye”. The image processing demonstrates that the scleral and/or conjunctiva blood vessel morphology can be used for discrimination and classification, either singly, or, more preferably in combination with morphological information in various spectral ranges.

Several advanced methods for classification of the images were applied, allowing more differentiation between infected and healthy patients by revealing hidden non-linear correlations. Additionally, a process for the detection of a single red blood cell and its movement was applied to a movie of conjunctival capillaries in a healthy volunteer. Image stabilization was applied using Speeded-Up Robust Features (SURF) image processing procedure which uses feature points. The matching features between the images was found, and, using affine transformation, the Mean Square Error was minimized. Single blood cells were tracked using a procedure that minimizes the bidirectional error. This time-dependent analysis provides an additional layer for quantification and classification of blood cell-related metrics.

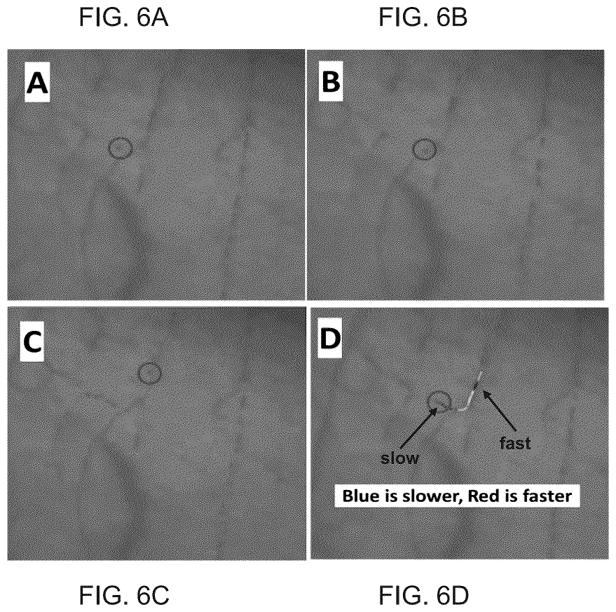

FIGS. 6A-D show detection and tracking of blood cells in conjunctival capillaries using the prototype system of the present embodiments. Due to the small width of the capillary only a single blood cell can travel in it. FIGS. 6A-C show analysis of a series of executive consecutive images. The Inventors have been able to detect and track a single red blood cell traveling from left to right and then up in one of these capillaries. Such information allows determining the velocity of RBCs in the capillaries and the densities of WBCs (gaps between RBCs). Examining the image with several different spectral ranges can provide specific relations to clinical parameters, e.g., oxygenated and non-oxygenated hemoglobin. FIG. 6D shows a velocity map of the red blood cell. Blue is slow (˜3.9 mm/sec), whereas Red is fast (˜16.9 mm/sec).

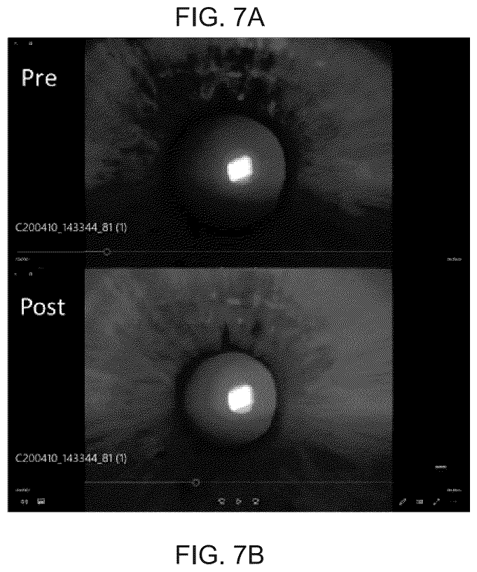

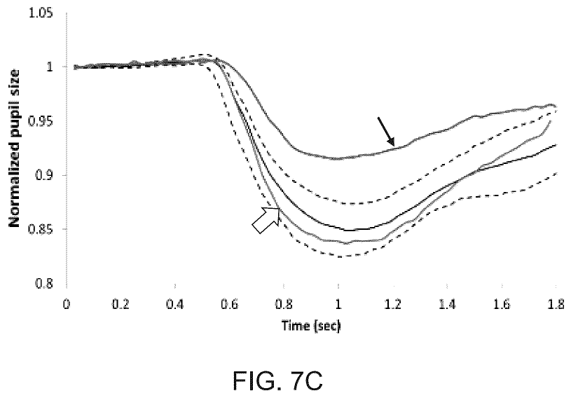

The ability to capture the PLR using the slit lamp imaging system in human subjects was also demonstrated by shining blue light. FIGS. 7A and 7B show pupil contraction in a healthy volunteer before (FIG. 7A) and after (FIG. 7B) chromatic light stimulus with blue light (about 500 ms). FIG. 7C shows attenuated pupil contraction in a representative subject with a brain tumor (red line) compared with age-similar controls (mean in a solid black line±SD in dashed lines) and its recovery following tumor removal (green line).

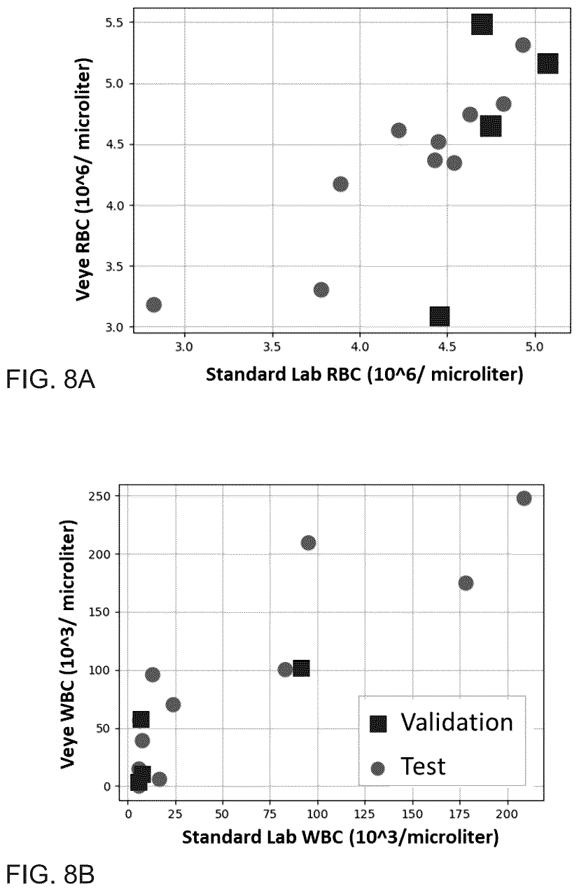

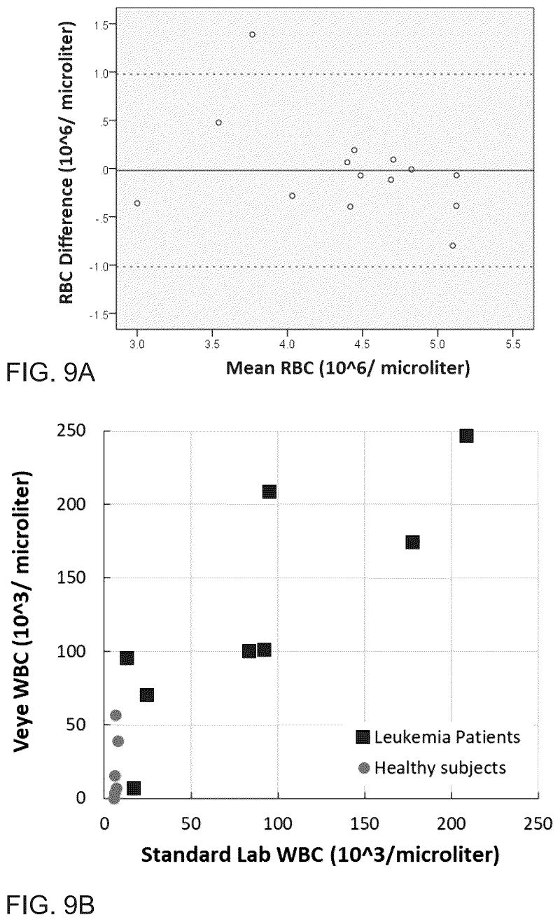

Following the application of Speeded-Up Robust Features (SURF) image-processing algorithm, which uses feature points, the matching features between the images were identified, and, using affine transformation, the Mean Square Error was minimized. Single blood cells were then tracked using an algorithm that minimizes the bidirectional error. FIGS. 8A-B show high correlation between red (FIG. 8A) and white (FIG. 8B) blood cell counts obtained by the imaging system (y-axis) and same day laboratory test results (x-axis). After algorithm training, graph represent data of patients in the “validation” group (circles) and “test” group (squares). This time-dependent analysis provides an additional layer for the quantification and classification of blood cells related metrics. FIGs. 9A-B show high accuracy of the testing by the system of the present embodiments. FIG. 9A show Bland Altman analysis demonstrating a high agreement between RBC counts obtained by imaging and laboratory results (mean difference=−0.029 10⁶ cell/microliter, p=0.88). FIG. 9B differentiates between leukemia patients (squares) and healthy subjects (circles) with high accuracy (ROC AUC=91.7%, p=0.01).

As shown in FIGS. 8A-B and 9A-B WBC and RBC counts obtained by imaging according to some embodiments of the present invention highly correlated with standard laboratory test results (Spearman rho=0.899, p=0.000012 and rho=0.798, p=0.001, respectively). ROC analysis demonstrated that the system of the present embodiments can differentiate between leukemia patients and healthy subjects with high accuracy (ROC AUC=91.7%, p=0.01, FIG. 9B)

### Discussion

This Example demonstrated the ability of the technique of the present embodiments to detect conjunctivitis and other diseases for example COVID-19, based on the simultaneous detection of systemic changes in neural, immune and vascular systems via quick imaging of the anterior segment of the eye. Those changes can be biomarkers for systemic or ocular diseases. Including blood count changes, hemodynamic changes, cardiovascular and vessels changes, pupil neurological and neuroretinal changes, conjunctivitis or any type of “red eye” or “yellow eye” (hepatitis) and glaucoma. The technique of the present embodiments combines simultaneous detection of systemic changes in neural, immune and vascular systems via quick imaging of the anterior segment of the eye.

### Example 2

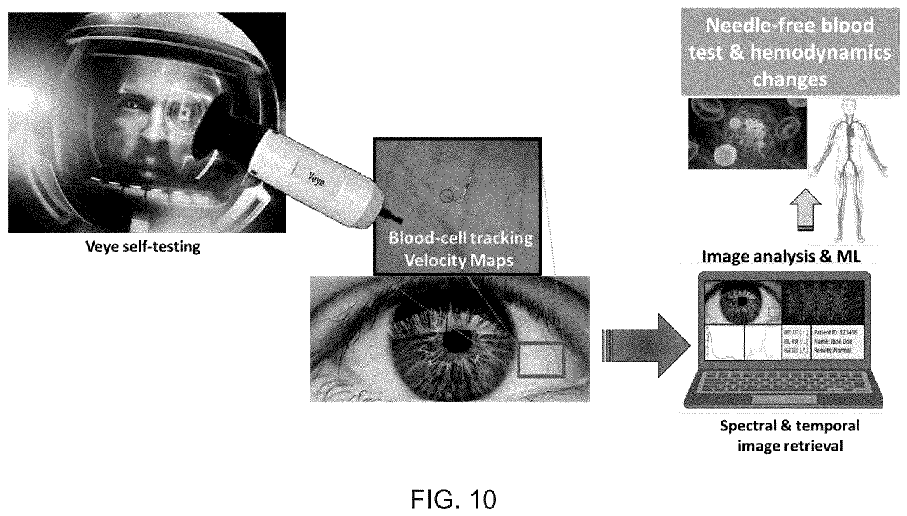

As humans gradually overcome technological challenges of deep space missions, the need for better understanding the physiological changes associated with long space-flights and for extraterrestrial remote healthcare solutions is on the rise. Clinical testing such as blood cell counts and blood oxygen levels that are routinely used on Earth for triage, diagnosis of numerous diseases and monitoring patient health, are still an unmet need in space. This Example describes a system, referred to as “Veye,” which is a portable multimodal imaging platform for point-of-care needle-free blood testing in space. The system captures high-resolution multispectral video images of the blood vessels at the front of the eye. The advantage of imaging these blood vessels is that they are the only blood vessels that are readily visible in the body with no masking of overlaying pigmented skin tissue or optical structures. The principles of the Veye system of the present embodiments are illustrated ion FIG. 10.

Spaceflights lead to various physiologic changes affecting nearly all the systems in the human body, including the cardiovascular system and the immune system. Microgravity, radiation, physical and psychological stressors, altered nutrition, disrupted circadian rhythms, and other factors cause immune system dysregulation manifested by increased white blood cell (WBC) count, persistent mild inflammation, infections and reactivation of latent herpesviruses. Currently, there is minimal clinical laboratory capability aboard the International Space Station, and the ability to monitor WBC during spaceflight is an unmet NASA medical requirement. Collecting blood samples in microgravity is a cumbersome and time-consuming analysis procedure that requires wearing a personal protective equipment to protect the astronauts from irritant solutions being used. Hence, this invasive complicated procedure is not applicable for routine and frequent monitoring of astronaut's WBC counts, and will not be feasible during deep-space missions.

Microgravity has an immediate effect on the cardiovascular system and leads to changes in many other hematological parameters. Without constant gravitation force, an almost immediate shift of fluids toward the head occurs, resulting in a “puffy” face and a reduced leg volume (“chicken legs”). An “acute plethora” of blood surrounds the central organs as peripheral blood is no longer held in the extremities by gravity. Studies suggest that red blood cell (RBC) and platelets counts and hemoglobin levels are elevated throughout space flights while plasma volume is decreased [Kunz et al., 2017]. However, the inventors found that these findings are extremely limited, as blood samples were collected either on Earth after landing, or shortly before return to Earth, significantly limiting the number of time points that could be tested in space. Moreover, as the cellular concentrations are dependent on plasma volume, the observed elevations may be influenced by dehydration or reduction in plasma volume in space without any real increase in cellular mass. Currently, there are no diagnostic tools that enable measuring these hemodynamic and hematologic changes frequently and non-invasively in space. Hence, the effect of space flights on RBC and platelet counts and hemoglobin levels remains largely unknown.

In addition, astronauts experience a decrease in blood vessel function during spaceflights. Decrease in cardiac output, blood volume and blood flow to skeletal muscles during space flight lowers oxygen uptake, decreases convective oxygen transport and oxygen diffusing capacity. Based on exercise measurements of astronauts before and after 6 months onboard the ISS, a dramatic reduction (30%-50%) was observed in maximal oxygen uptake [Ade et al., 2017], the maximum rate of oxygen consumed during exercise and shows the cardiorespiratory health of a person. As astronauts have to perform many physically demanding tasks on board the ISS, as well as life-saving tasks when they return to gravity (e.g., during emergency landing on Earth or performing extravehicular activities planned on the surface of the Moon and Mars), monitoring oxygen blood level is advantageous for monitoring astronauts' health and providing interventions if needed. Lower tissue oxygen levels may also accelerate osteoporosis. Oxygen saturation is routinely determined on Earth using commercially available pulse oximeters clipped onto the patient's fingertip. This technology is based on near-infrared spectroscopy, exploiting oxygenated and deoxygenated hemoglobin's characteristic light absorption properties in the near-infrared wavelength range. However, it is recognized that pulse oximeters overestimate blood-oxygen saturation three times more frequently in Afro-Americans than Caucasians due to interference by dark skin. This racial bias may have been one of the reasons for the higher mortality rate in the Afro-American and Latin populations during the COVID-19 pandemic. This bias may also affect the accuracy of blood oxygen level measurements of non-Caucasian astronauts currently done onboard the ISS using wearable systems such as Bio-Monitor which uses a wearable headband oximeter which may be affected by skin-color.

During planned deep space missions to the moon and beyond, the stressors that cause immune system dysregulation, decrease in blood vessel function and hematological changes will increase, while clinical care capability options for various biomedical countermeasures will likely be reduced. Hence, there is an unmet need for diagnostic tools that can provide a point-of-care assessment of these in-flight blood changes.

In recent years, eye imaging has emerged as a tool offering a window that goes beyond the diagnosis of ophthalmic conditions. Indeed, traditional imaging methods of the visual system are mainly focused on the identification of anatomical changes in the back (posterior) part of the eye (mainly retina and optic nerve) using fundoscopy, slit lamp exams, optical coherence tomography (OCT), ocular and optic nerve ultrasound, and MRI. When imaging the front (anterior) part of the eye, ultrasound biomicroscopy, corneal pachymetry, and anterior-segment OCT are commonly used in the clinic. These techniques, however, focus on the angle and corneal structure or thickness. The present Example describes ocular imaging modalities for the diagnosis and monitoring of eye and brain pathologies. The technique allows objective assessment of corneal lesions, corneoscleral thinning and microarchitecture of Schlem canal. The technique can also monitor retinal degeneration, and ocular imaging for early diagnosis of Alzheimer disease and brain tumors.

The Inventors found that assessment of changes in the microvasculature at the front part of the eye provides a unique window to identify changes in blood flow, plasma volume, blood cell count, hemoglobin, and blood-oxygen saturation levels.

The system described herein is a portable multimodal-imaging platform for point-of-care needle-free blood testing in space. The system captures short (typically less than 5 or less than 4 or less than 3 or less than 2 minutes) high frequency, high-resolution video images of the capillaries at the front of the eye. The system is particularly useful for self-testing, and combines spectral and temporal sectioning methods, as well as Artificial Intelligence methods. The system can be used for monitoring various hematologic and hemodynamic parameters, including, without limitation, changes in platelets, red, and white blood cells, blood flow, hemoglobin and oxygen saturation levels in space. As the blood vessels at the front of the eye are the only readily-visible blood vessels in the human body, with no masking of overlaying pigmented tissues, the system described herein provides blood test results with no racial bias. Image analysis can provide information pertaining to the astronauts' neurological, ocular, hemodynamic and cardiovascular condition. It is predicted that the analysis will provide information regarding the effect of space flight on human physiology.

In some embodiments of the present invention the system includes a headset configured to place the camera in front of the eye.

Preferably the imaging is executed in less than two minutes, so as to allow frequent monitoring of physiological changes.

In some embodiments of the present invention the camera is equipped with automatic focusing for better quantification of the temporal and morphological changes for different wavelengths. The astronauts' eyes can be imaged several times per day, for example, three times per day (e.g., morning, noon and evening), on two or more consecutive days before leaving Earth, while being outside the Earth's atmosphere (e.g., in a space station), and optionally and preferably also after returning to Earth. Data can be sent to a data processor, e.g., on Earth, for processing.

In narrow capillaries, the capillary diameter approaches WBC diameter (about 10-20 μm), so that the WBC fills the capillary lumen. Since the velocity of WBCs is slower than that of RBCs, a depletion of RBCs occurs downstream of the WBC in the microcirculation [Schmid-Schönbein, 1980]. Illuminating blood vessels with light enables to detect RBCs that look dark as they absorb the light, whereas WBCs stay transparent. Thus, the passage of a WBC appears as an optical absorption gap in the continuous dark RBC stream that moves through the capillary. This was shown in rabbit ears using white light. In rat-cremaster, bat-wings and human nailfold capillaries, blue light trans-illumination maximizes the contrast between high-absorbing RBCs (that carry large amounts of hemoglobin) and low-absorption regions lacking RBCs. However, since nailfold's capillaries are masked by the skin, this method is applicable only for people with light skin (typically less than 4 on the Fitzpatrick scale). Absorption gaps can also be observed in the retina using adaptive optics scanning laser ophthalmoscopy (AO-SLO). However, due to its high cost, prolonged data acquisition time, and highly trained personnel required for image capture and analysis, the AO-SLO technology remains a research tool. Furthermore, imaging retinal blood vessels requires pupil dilation, and imaging may be hindered by common conditions such as cataract that blocks the optic pathway.

The optical properties of blood components vary considerably. While RBC's absorption spectra is dominated by the optical properties of hemoglobin, WBCs have a peak absorbance in IR and UV ranges, and platelets at about 450 nm and about 1000 nm. Oxygenated hemoglobin (HbO₂) has different light-absorption spectra than deoxygenated hemoglobin (Hb), between about 600 nm and about 1000 nm, which can be used to differentiate between them. Pulse oximetry devices pass two wavelengths of light, typically about 660 nm and 940 nm, through the skin (commonly fingernail) to a photodetector that measures the changing absorbance at each wavelength. As the absorption of light at these wavelengths differs significantly between blood loaded with oxygen and blood lacking oxygen, the saturation of peripheral oxygen (SpO₂ or SaO₂) can be determined, according to SaO₂=[HbO2]/([HbO2]+[Hb]). Yet, known oximetry devices are, as stated, biased due to interference by dark skin.

The Inventors found that multispectral imaging of the highly accessible narrow microvascular blood vessel at the front of the eye (limbus or conjunctiva) allows fast (e.g., real-time) non-invasive detection of dynamic spatial and temporal changes in blood components, including oxygen levels, in different selected capillaries, for clinical diagnosis of various pathological conditions.

The Inventors found that deep neural networks (DNNs) can provide a unique, robust, time-efficient and accurate characterization capability for complex structures based on their far-field optical responses. The system described herein uses DNN to address the high level of nonlinearity of inference tasks by creating a model that holds bidirectional knowledge. Infrared imaging offers an analytical tool owing to the organic materials' fingerprints in this region of the electromagnetic spectrum. However, sensors in this region are slow, low resolution, expensive and require cooling. Commercially available infrared sensors also fail to provide instantaneous multicolor imaging. The system of the present embodiments optionally and preferably uses an adiabatic nonlinear crystal for performing up-conversion imaging. The advantage of these embodiments is that they provide high resolution, fast, morn temperature and multicolor imaging of MWIR scenes. This can also provide remote sensing of chemicals and organic compounds.

The system described herein combines spatial and temporal imaging at the visible-near-IR with correlation analysis and machine learning methods. The system optionally and preferably employs a hyperspectral camera. Multispectral imaging of the retina have been suggested [Kaluzny et al., 2017] for determining vascular oxygen saturation imaging. However, the inventors found that there are several drawbacks in this technique. These include (i) masking of overlaying tissues such as opacities in cornea, lens or vitreous, (ii) variability in Retinal Pigment Epithelium pigmentation, (iii) chromatic aberrations, (iv) variability in the structure of the eye between subjects, (v) variability in the amount of light penetrating the eye if mydriatics (dilating eye drops) are not used. Unlike the convectional retinal imaging, the technique of the present embodiments capture images of the front of the eye and leverages the wealth of the hyperspectral data to provide dynamical analysis. It is expected that the redundancy of data for a given specimen is between 10 to 24 spectral lines. Such redundancy effectively augment the available dataset and can be exploited according to some embodiments of the present invention by machine learning, such as, but not limited to, deep learning, procedures.

The system described herein allows the retrieval of multi-spectral video images of capillaries across the Visible-Near-infrared. These images, illuminated and detected in different spectral ranges, along with the time-dependent tracking and analysis of specific images' features, offer a rich dataset for various types of analysis, optionally and preferably, but not necessarily by means of machine-learning. The system allows fast detection of subtle changes in the libmal or conjunctival and limbal capillaries blood vessel hemodynamics at various relevant spectral wavelengths. Preferable the detection is in real time.

The system of the present embodiments optionally and preferably comprises a portable multi-spectral imaging system for retrieving high-resolution eye scans at VIS-NIR spectral range, allowing both spectral and spatial information retrieval. The system is preferably compatible with space transportation and ISS Standards.

The system of the present embodiments preferably employs a machine learning analysis and classification procedures to associate the laboratory test blood results and conventional oximetry measures with microvascular features in healthy subjects and patients with hematologic diseases on Earth. The procedures can be trained for diagnostic accuracy of longitudinal spatio-temporal changes in the limbal or conjunctival capillaries associated with disease progression, response to treatment, and relapse or deterioration, taking into account individual variability.

The system of the present embodiments can be used to characterize the effect on space flight on blood cell counts and hemodynamics. The system of the present embodiments can be use for establishing a dataset of clinical data, by monitoring the astronauts in microgravity conditions. The dataset can optionally and preferably be used for updating the data analysis procedures.

The system described herein allows point-of-care assessment of the severity of astronauts' medical conditions during space missions. The system can allow early intervention and frequent tracking of responses to treatment. The system can allow detection and/or diagnoses of many medical conditions, such as, but not limited to, the in-flight medical conditions listed on NASA Exploration Medical Condition List, particularly, but not exclusively, infections, acute radiation syndrome, and potential surgical conditions, such as, but not limited to, appendicitis or cholecystitis. The point-of-care capability of the system of the present embodiments can provide crew health data-point to augment traditional measures, which may be especially useful with increased spaceflight hazards during future deep space missions. The system of the present embodiments can allow astronauts and medical teams to make better informed medical decisions and improve the ability to monitor, diagnose, and treat astronauts in space. Moreover, the clinical data collected from the astronauts before, during- and following the space mission can be used to enhance the understanding of the physiological changes that occur during space flights. These findings may impact the design of future space missions and may lead to the development of new space-relevant interventions.

The system described herein can also allow point-of-care needle-free diagnosis of hematologic conditions on Earth. Such conditions may include blood cell cancers, anemia, and complications from chemotherapy or radiotherapy. This is particularly advantageous for diagnosing and monitoring patients with limited mobility (elders, handicapped, babies etc.), in remote and medically underserved locations. In particular, means for fast non-invasive diagnosis, including blood hemodynamics, cell blood count, blood-oxygen level and hemoglobin levels, are still missing for meaningful remote care, as emphasized by the current COVID-19 outbreak. On Earth, the system of the present embodiments can reduce office and emergency room visits can therefor allows fast and frequent testing for continuous monitoring of patient deterioration or recovery and response to treatment with no skin color bias. The system of the present embodiments can improve the survival and quality of life of millions of patients routinely undergoing blood tests for assessment of their general health, immune status and cancer diagnosis, including newborns, patients with blood cancer, patients undergoing chemotherapy, and critically ill patients.

The system of this example includes as high-speed multi-spectral imaging system for retrieving high-resolution eye scans at VIS-NIR spectral range allowing both spectral and spatial information retrieval. In some embodiments of the present invention system is a portable hand-held system. Preferably, the multi-spectral imaging system is configured to capture images at several wavelengths, for example, about 540 nm, about 660 nm and about 940 nm for red blood cells and oxygenated and deoxygenated hemoglobin, respectively, and about 450 nm for platelets. The light intensity and frame rate is preferably selected to allow capturing of blood flow in vessels. In experiments performed according to some embodiments of the present invention images were acquired at 30 frames per second, namely the exposure time of about 33 ms per frame. During this exposure time, the blood cell movements were visible.

In some embodiments of the present invention the illumination is by successive short flashes, thus capturing a shorter time segment within the overall long exposure time. This allows capturing images with more blood cells. For example, when using 5 ms flashes at sufficient illumination, the effective exposure time is about 1 ms even when the exposure is about 30 ms. In some embodiments of the present invention the system includes a display for live presentation of the images being captured, to allow the user to focus the image on his own blood vessels. In experiments performed by the Inventors, following a short (5 minutes) training, an astronaut successfully focused the image on the display screen and captured high-quality video images of the blood vessel on the front part of his eyes.

In an exemplified study, an Earth clinical database is collected using the system of the present embodiments. The clinical database is collected, on Earth, from patients with hematologic conditions that have aberrant blood counts and healthy controls on. Eyes of 20 leukemia patients with abnormal high white blood cell counts, 20 patients with very low white blood cell counts (Neutropenia) due to high-dose chemotherapy, 20 patients with polycythemia vera (abnormal high RBC count), 20 patients with severe anemia (low levels of hemoglobin) and 200 age- and gender-similar controls are imaged utilizing the system of the present embodiments. Age, gender, smoking and medications are recorded, as well as the date of diagnosis for the patients. Body temperature, intraocular pressure (IOP), blood oximetry, systolic and diastolic blood pressure and heart rate are measured in all study participants at the time of imaging. Blood samples are collected from all subjects on the same day of imaging for complete blood count, hemoglobin levels and hematocrit. Main general inclusion criteria are non-pregnant adults (>18 YO) that can understand and sign a consent form. Main general exclusion criteria are recent (3 months) or ongoing eye diseases, eye drop treatment or use of local sympathomimetic or para-sympatholytic medications prior to eye imaging, pregnancy. 200 Healthy subjects (no past or current cancer or hematologic disorder), age similar to patients, with a leukocyte count 3,000-11,000 cells/μL and a neutrophil count ≥1,500 cells/μL, are used as the control group. Exclusion criteria for the controls include, in addition to the general exclusion criteria, severe stress or anxiety, known anemia, current allergy attack, asthma, fever. The Patient group includes: (1) leukemia patients—20 Chronic Lymphocytic Leukemia (CLL) patients with leukocytosis with >50,000 WBC/μL on the same day of imaging; (2) Neutropenia patients—20 cancer patients receiving chemotherapy at the severe neutropenia stage with <500 neutrophils/μL on the same day of imaging; (3) Polycythemia patients—subjects with a diagnosis of primary or secondary Polycythemia with abnormal high RBC before treatment onset; and (4) Patients with Anemia—20 patients with moderate to severe anemia (Hemoglobin ≤10.0 g/dL). To assess the accuracy for the detection of patient recovery, relapse, or deterioration, accounting for individual variability, a longitudinal follow-up testing (at least six repeated testing) is performed for at least 10 subjects from each study group. Controls and Leukemia patients are tested at least six times, once a week. In the Neutropenia group, cancer patients are tested prior to receiving first dose of chemotherapy (baseline). and then once a week following chemotherapy treatment for five additional weeks. The majority of patients suffer from neutropenia 1-2 weeks after treatment (Nadir) and by 3-4 weeks neutrophil count returns to a normal level. Polycythemia patients and patients with anemia are tested before treatment and at least five times every two weeks after treatment. Based on a correlation coefficient of 0.798 obtained by the Inventors in preliminary studies between RBC counts as obtained by imaging, and standard laboratory blood analysis results, with α=0.05, 1−13=0.95, a sample size of 7 subjects is selected (G*power software). A larger sample size of 20 patients per group is used for clinical significance and to cover a wide range of WBC and RBC counts. The size of the dataset is sufficient for the ML taking note that each frame of the movies constitutes a sample with respect to the dataset.

The collected data are analyzed in two phases. In a first phase an image processing procedure is applied to retrieve subtle changes in the conjunctival and/or limbal blood vessel morphology and blood flow. The image processing is applied in several spectral lines to differentiate various biological markers, such as, but not limited to, oxygen, red- and white-blood cell densities, etc. In a second phase, machine learning procedure is applied for detection and classification. The machine learning procedure treats the image as a whole and is natively inclined to reveal nonlinear correlations leading to much improved classification in the multidimensional dataset. The machine learning procedure can include at least one of principal component analysis (PCA), support vector machine (SVM), and a deep neural network (DNN) such as, but not limited to, GaN and pix2pix networks. The pix2pix algorithm, which operates successful image-to-image translation, has been shown to perform particularly well for the classification of images where there is a low separation between the different classes and difficulty in learning discriminative features. Analysis of moving blood cells in the libmal or conjunctival capillaries is also employed for non-invasive fast detection of clinical parameters such as WBC and hemoglobin. This approach complements and further augment the classification by machine learning.

For implementation of the system in space, astronauts can undergo self-testing within 24 hours before launch, daily on board the ISS, and within 24 hours after landing. Data can be transferred daily to Earth for analysis. Blood samples can be collected on Earth and approximately hours prior to hatch closure of the returning vehicle for blood cell count. Laboratory test blood results and oximetry measures on Earth can be associated with the microvascular features detected by the system of the present embodiments, and changes at different stages of the space mission (e.g., pre-during-post) can be determined.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] van Kasteren, P. B. et al. Comparison of seven commercial RT-PCR diagnostic kits for COVID-19. J. Clin. Virol. 128, 104412 (2020).
[2] Wang, D. et al. Clinical Characteristics of 138 Hospitalized Patients With 2019 Novel Coronavirus-Infected Pneumonia in Wuhan, China. JAMA (2020). doi: 10.1001/jama.2020.1585.
[3] Tan, L. et al. Lymphopenia predicts disease severity of COVID-19: a descriptive and predictive study. medRxiv (2020). doi:10.1101/2020.03.01.20029074.
[4] Wu, P. et al. Characteristics of Ocular Findings of Patients With Coronavirus Disease 2019 (COVID-19) in Hubei Province, China. JAMA Ophthalmol. (2020). doi: 10.1001/jamaophthalmol.2020.1291.
[5] Lauande, R. & Paula, J. S. Coronavirus and the eye: what is relevant so far? Arq Bras Oftalmol 83, V-VI (2020).
[6] Mao, L. et al. Neurologic manifestations of hospitalized patients with coronavirus disease 2019 in wuhan, china. JAMA Neurol. (2020). doi:10.1001/jamaneurol.2020.1127.
[7] Menni, C. et al. Real-time tracking of self-reported symptoms to predict potential COVID-19. Nat. Med. (2020). doi:10.1038/s41591-020-0916-2.
[8] Spinato, G. et al. Alterations in Smell or Taste in Mildly Symptomatic Outpatients With SARS-CoV-2 Infection. JAMA (2020). doi:10.1001/jama.2020.6771.
[9] Baig, A. M., Khaleeq, A., Ali, U. & Syeda, H. Evidence of the COVID-19 Virus Targeting the CNS: Tissue Distribution, Host-Virus Interaction, and Proposed Neurotropic Mechanisms. ACS Chem. Neurosci. 11, 995-998 (2020).
[10] Li, Y.-C., Bai, W.-Z. & Hashikawa, T. The neuroinvasive potential of SARS-CoV2 may play a role in the respiratory failure of COVID-19 patients. J. Med. Virol. (2020). doi: 10.1002/jmv.25728.
[11] Choudhary, R., Kapoor, M. S., Singh, A. & Bodakhe, S. H. Therapeutic targets of renin-angiotensin system in ocular disorders. Journal of Current Ophthalmology 29, 7-16 (2017).
[12] Senanayake, P. deS et al. Angiotensin II and its receptor subtypes in the human retina. 48, 3301-3311(2007).
[13] London, A., Benhar, I. & Schwartz, M. The retina as a window to the brain-from eye research to CNS disorders. Nat. Rev. Neurol. 9, 44-53 (2013).
[14] Giza, E., Fotiou, D., Bostantjopoulou, S., Katsarou, Z. & Karlovasitou, A. Pupil light reflex in Parkinson's disease: evaluation with pupillometry. Int. J. Neurosci. 121, 37-43 (2011).
[15] Samadzadeh, S., Abolfazli, R., Najafinia, S., Morcinek, C. & Rieckmann, P. Quantitative manual pupillometry as a valuable tool to assess visual pathway function in Multiple Sclerosis: first results on potential association with fatigue (P4.413). 90, (2018).
[16] Chen, J. W. et al. Pupillary reactivity as an early indicator of increased intracranial pressure: The introduction of the Neurological Pupil index. Surg Neurol Int 2, 82 (2011).
[17] Taylor, W. R. et al. Quantitative pupillometry, a new technology: normative data and preliminary observations in patients with acute head injury. Technical note. J. Neurosurg. 98, 205-213 (2003).
[18] Bittner, D. M., Wieseler, I., Wilhelm, H., Riepe, M. W. & Müller, N. G. Repetitive pupil light reflex: potential marker in Alzheimer's disease? J. Alzheimers Dis. 42, 1469-1477 (2014).
[19] Frost, S. M. et al. Pupil response biomarkers distinguish amyloid precursor protein mutation carriers from non-carriers. 10, 790-796 (2013).
[20] Fotiou, D. F. et al. Cholinergic deficiency in Alzheimer's and Parkinson's disease: evaluation with pupillometry. Int. J. Psychophysiol. 73, 143-149 (2009).
[21] Nyström, P., Gredebäck, G., Bölte, S., Falck-Ytter, T. & EASE team. Hypersensitive pupillary light reflex in infants at risk for autism. Mol. Autism 6, 10 (2015).
[22] Hall, C. A. & Chilcott, R. P. Eyeing up the future of the pupillary light reflex in neurodiagnostics. Diagnostics (Basel) 8, (2018).
[23] Wainstein, G. et al. Pupil Size Tracks Attentional Performance In Attention-Deficit/Hyperactivity Disorder. Sci. Rep. 7, 8228 (2017).
[24] Rotenstreich, Y. et al. Chromatic multifocal pupillometer for objective diagnosis of neurodegeneration in the eye and the brain. Invest. Ophthalmol. Vis. Sci. (2016).
[25] Bourquard, A. et al. Non-invasive detection of severe neutropenia in chemotherapy patients by optical imaging of nailfold microcirculation. Sci. Rep. 8, 5301 (2018).
[26] McKay, G. N. et al. Visualization of blood cell contrast in nailfold capillaries with high-speed reverse lens mobile phone microscopy. Biomed. Opt. Express 11, 2268-2276 (2020).
[27] Chibel, R. et al. Chromatic Multifocal Pupillometer for Objective Perimetry and Diagnosis of Patients with Retinitis Pigmentosa. Ophthalmology 123, 1898-1911 (2016).
[28] Haj Yahia, S. et al Effect of Stimulus Intensity and Visual Field Location on Rod- and
Cone-Mediated Pupil Response to Focal Light Stimuli. Invest. Ophthalmol. Vis. Sci. 59, 6027-6035 (2018).
[29] Sher, I. et al. and Rotenstreich Y. Chromatic pupilloperimetry measures correlate with visual acuity and visual field defects in retinitis pigmentosa patients. TVST (Accepted, 2020).
[30] Miyake, H., Oda, T., Katsuta, O., Seno, M. & Nakamura, M. A Novel Model of Meibomian
Gland Dysfunction Induced with Complete Freund's Adjuvant in Rabbits. Vision 1, 10 (2017).
[31] Bharathi, R. D. & Kumar, B. V. Automatic Detection of Adenoviral Disease from Eyes Images Using HOG Technique.
[32] Gunay, M., Goceri, E. & Danisman, T. Automated Detection of Adenoviral Conjunctivitis Disease from Facial Images using Machine Learning. in 2015 IEEE 14th International Conference on Machine Learning and Applications (ICMLA) 1204-1209 (IEEE, 2015). doi:10.1109/ICMLA.2015.232
[33] Gumus, E., Kilic, N., Sertbas, A. & Ucan, 0. N. Evaluation of face recognition techniques using PCA, wavelets and SVM. Expert Syst. Appl. 37, 6404-6408 (2010).
[34] Isola, P., Zhu, J.-Y., Zhou, T. & Efros, A. A. Image-to-Image Translation with Conditional Adversarial Networks. in 2017 IEEE Conference on Computer Vision and Pattern Recognition (CVPR) 5967-5976 (IEEE, 2017). doi:10.1109/CVPR.2017.632.
[35] Wang, X., Yan, H., Huo, C., Yu, J. & Pant, C. Enhancing pix2pix for remote sensing image classification. in 2018 24th International Conference on Pattern Recognition (ICPR) 2332-2336 (IEEE, 2018). doi:10.1109/ICPR.2018.8545870.
[36] Rotenstreich, Y. et al. Treatment with 9-cis (3-carotene-rich powder in patients with retinitis pigmentosa: a randomized crossover trial. JAMA Ophthalmol. 131, 985-992 (2013).
[37] Rotenstreich, Y. et al. Association of brain structure and cognitive function with structural retinal markers in asymptomatic individuals at high risk for Alzheimer disease. 60, 1878 (2019).
[38] Sher, I. and Rotenstreich Y. Repetitive magnetic stimulation protects corneal epithelium in a rabbit model of short-term exposure keratopathy. Ocul Surf 18, 64-73 (2020).
[39] Malkiel, I. et al Plasmonic nanostructure design and characterization via Deep Learning. Light Sci Appl 7, 60 (2018).
[40] Malkiel, I., Mrejen, M., Wolf, L. & Suchowski, H. Spectra2pix: Generating Nanostructure Images from Spectra. arXiv (2019).
[41] Malkiel, I., Mrejen, M., Wolf, L. & Suchowski, H. Machine learning for nanophotonics. MRS Bull. 45, 221-229 (2020).
[42] Kunz H, Quiriarte H, Simpson R J, et al. Alterations in hematologic indices during long-duration spaceflight. BMC Hematol 2017; 17:12.
[43] Ade C J, Broxterman R M, Moore A D, Barstow T J. Decreases in maximal oxygen uptake following long-duration spaceflight: Role of convective and diffusive 02 transport mechanisms. J Appl Physiol 2017; 122:968-975.
[44] Smith T G, Formenti F, Hodkinson P D, et al. Monitoring tissue oxygen saturation in microgravity on parabolic flights. Gravitational and Space Research 2016; 4:2-7.
[45] Schmid-Schönbein G W, Usami S, Skalak R, Chien S. The interaction of leukocytes and erythrocytes in capillary and postcapillary vessels. Microvasc Res 1980; 19:45-70.
[46] Sinclair S H, Azar-Cavanagh M, Soper K A, et al. Investigation of the source of the blue field entoptic phenomenon. Invest Ophthalmol Vis Sci 1989; 30:668-673.
[47] Kaluzny J, Li H, Liu W, et al. Bayer filter snapshot hyperspectral fundus camera for human retinal imaging. Curr Eye Res 2017; 42:629-635.
[48] Liu K-Z, Xu M, Scott D A. Biomolecular characterisation of leucocytes by infrared spectroscopy. Br J Haematol 2007; 136:713-722.
[49] Terent'yeva Y G, Yashchuk V M, Zaika L A, et al. The manifestation of optical centers in UV-Vis absorption and luminescence spectra of white blood human cells. Method Appl Fluoresc 2016; 4:044010.
[50] Kim J G, Xia M, Liu H. Extinction coefficients of hemoglobin for near-infrared spectroscopy of tissue. IEEE Eng Med Biol Mag 2005; 24:118-121.
[51] Meinke M, Müller G, Helfmann J, Friebel M. Optical properties of platelets and blood plasma and their influence on the optical behavior of whole blood in the visible to near infrared wavelength range. J Biomed Opt 2007; 12:01402.
[52] Arichika et al., Investigative Ophthalmology & Visual Science June 2013, Vol. 54, 4394-4402.
[53] Uji et al., Invest Ophthalmol Vis Sci. 2012 Jan. 20; 53(1):171-8.

What is claimed is:

1. A method of diagnosing a condition of a subject, comprising:
receiving a stream of image data of an anterior of an eye of the subject at a rate of at least frames per second;
applying a spatio-temporal analysis to said stream to detect flow of individual blood cells in limbal or conjunctival blood vessels of said eye;
based on detected flow, determining the condition of the subject.

2. The method according to claim 1, comprising identifying hemodynamic changes in the body of the subject based on said detected flow.

3. The method according to claim 1, wherein said spatio-temporal analysis comprises applying a machine learning procedure.

4. The method according to claim 1, wherein said image data comprise at least one monochromatic image.

5. The method according to claim 1, wherein said applying said spatio-temporal analysis is selected to identify pupil light reflex events, wherein said determining the condition is based also on said identified pupil light reflex events.

6. The method according to claim 1, wherein said applying said spatio-temporal analysis is selected to detect in said eye morphology of limbal or conjunctival blood vessels, wherein said determining the condition is based also on said detected morphology.

7. The method according to claim 1, comprising identifying flow of gaps.

8. The method according to claim 7, comprising measuring at least one of a size of said gaps and a flow speed of said gaps.

9. The method according to claim 1, wherein said detecting said flow is in at least two different vessels structures.

10. The method according to claim 1, comprising determining a density of said limbal or conjunctival blood vessels.

11. A method of diagnosing a condition of a subject, comprising:
receiving image data of an anterior of an eye;
applying a spectral analysis to said image data to detect in said morphology of limbal or conjunctival blood vessels;
based on said morphology, determining the condition of the subject.

12. The method according to claim 11, wherein said image data comprises a set of monochromatic images, each being characterized by a different central wavelength.

13. The method according to claim 11, wherein said image data is a stream of image data at a rate of at least 30 frames per second.

14. The method according to claim 1, wherein said image data comprises at least one multispectral image.

15. The method according to claim 1, comprising capturing said image data.

16. A method of diagnosing a condition of a subject, comprising:
receiving input pertaining to a wavelength that is specific to the subject, and that induces pupil light reflex in a pupil of the subject;
illuminating said pupil with light at said subject-specific wavelength;
imaging an anterior of an eye of the subject at a rate of at least 30 frames per second to provide a stream of image data;
applying a spatio-temporal analysis to said stream to detect pupil light reflex events; and
based on detected pupil light reflex events, determining the condition of the subject.

17. The method according to claim 16, wherein said spatio-temporal analysis is selected to detect flow of individual blood cells in libmal or conjunctival blood vessels of said eye, and wherein said determining the condition is also based on said detected flow.

18. The method according to claim 1, wherein the condition is selected from the group consisting of a disease, a bacterial disease, a viral disease, a coronavirus disease, sepsis, heart failure, an ischemic condition, cardiovascular disease, hematological disease, glaucoma, leukemia, a neuronal attenuation, anemia, neutropenia, polycythemia, jaundice, conjunctivitis, and any other condition or disease that affects blood content count and blood vessel flow.

19. The method according claim 1, comprising generating an output describing the condition in terms of at least one parameter selected from the group consisting of white blood cells count, red blood cells count, platelets count, hemoglobin level, oxygenated hemoglobin level, deoxygenated hemoglobin level, methemoglobin level, capillary perfusion, ocular inflammation, blood vessel inflammation, and blood flow.

20. A system for diagnosing a condition of a subject, comprising:
an imaging system for capturing image data of an anterior of an eye of the subject; and
an image control and processing system configured for applying the method according to claim 1.
